(19) United States
(12) Patent Application Publication
(10) Pub. No.: US 2026/0183063 A1
(43) Pub. Date: Jul. 2, 2026

(54) REGISTRATION OF PRE-OPERATIVE MEDICAL IMAGES USING INTRA-OPERATIVE MEDICAL IMAGES FOR IMPROVED VISUALIZATION ON AUGMENTED REALITY DISPLAY

(71) Applicant: Augmedics Ltd., Yokneam Illit (IL)

(72) Inventor: Gwen Watanabe, Dallas, TX (US)

(21) Appl. No.: 19/426,344

(22) Filed: Dec. 19, 2025

Related U.S. Application Data
(60) Provisional application No. 63/740,895, filed on Dec. 31, 2024.

(51) Int. Cl. A61B 34/20; A61B 34/10; A61B 90/00; G06T 7/11; G06T 7/33; G06T 17/20; G06T 19/00

(52) U.S. Cl. CPC A61B 34/20; A61B 34/10; A61B 90/37; G06T 7/11; G06T 7/337; G06T 17/20; G06T 19/006; A61B 2034/105; A61B 2090/365; A61B 2090/372; G06T 2207/10081; G06T 2207/10088; G06T 2207/30012; G06T 2207/30204; G06T 2210/41

(57) ABSTRACT

The disclosure generally relates to systems, devices and methods to facilitate image-guided medical treatment and/or diagnostic procedures (e.g., augmented reality guided surgery or other intervention among other considered medical usages), and to the generation of current and/or accurate anatomical images for facilitating image-guided medical treatment and/or diagnostic procedures and calibration and/or registration of imaging modalities used in such medical treatment and/or diagnostic procedures.

### CROSS-REFERENCE TO RELATED APPLICATIONS

This Application claims benefit of U.S. Provisional Patent Application Ser. No. 63/740,895, filed Dec. 31, 2024, the entire contents of which are hereby incorporated by reference. This Application is related to International PCT Publication No. WO 2024/018368, filed Jul. 17, 2023, and published on Jan. 25, 2024, titled “Calibration and Registration of Pre-Operative and Intraoperative Images”, which is hereby incorporated herein by reference in its entirety.

### FIELD

The disclosure generally relates to systems, devices, and methods to facilitate image-guided medical treatment and/or diagnostic procedures (e.g., augmented reality guided surgery or other intervention among other considered medical usages), and further relates to the generation of current and/or accurate anatomical images for facilitating image-guided medical treatment and/or diagnostic procedures (e.g., surgery or other intervention) and calibration and/or registration of imaging modalities (e.g., pre-operative 3D imaging and intra-operative 3D imaging) used in such medical treatment and/or diagnostic procedures.

### BACKGROUND

Image-guided surgery or medical procedures employ tracked surgical tools or instruments and images of the patient anatomy in order to guide the procedure. In such procedures, a proper and current imaging or visualization of regions of interest of the patient anatomy is of high importance.

Near-eye display devices and systems, such as head-mounted displays including special-purpose eyewear (e.g., glasses), are used in augmented reality (AR) systems.

See-through displays (e.g., displays including at least a portion which is see-through) are used in augmented reality systems, for example for performing image-guided and/or computer-assisted surgery. Typically, but not necessarily, such see-through displays are near-eye displays (e.g., integrated in a head-mounted device (HMD)). In this way, a computer-generated image may be presented to a healthcare professional who is performing the procedure, such that the image is aligned with an anatomical portion of a patient who is undergoing the procedure. Systems of this sort for image-guided surgery are described, for example, in U.S. Pat. Nos. 9,928,629, 10,835,296, 10,939,977, PCT International Publication WO 2019/211741, U.S. Patent Application Publication 2020/0163723, PCT International Publication WO 2022/053923, PCT International Publication WO 2023/203521, and PCT International Publication WO 2024/057210. The disclosures of all these patents and publications are incorporated herein by reference in their entirety.

### SUMMARY

In image-guided medical procedure navigation (e.g., augmented reality-assisted spinal navigation), a computing system (e.g., one or more processors with associated memory) typically receives intra-operative medical imaging (e.g., 3D images) of a portion of an anatomy of a patient (e.g., a spine or portion of a spine, another orthopedic joint such as a hip, knee, shoulder, ankle, or jaw, or other patient anatomy) captured by an imaging modality (e.g., computed tomography (CT) scanner or imaging device or magnetic resonance imaging (MRI) scanner or imaging device, an ultrasound imaging device, or other medical imaging devices). The computing system then registers the intra-operative medical imaging to one or more fiducials (e.g., markers) attached to or placed on the patient. After registration, medical instruments for performing a medical intervention (e.g., therapeutic procedure or diagnostic procedure) that also include a trackable marker can be tracked relative to the patient (e.g., the one or more fiducials) and the position of the medical instrument(s) can be displayed using virtual images or icons of the medical instruments with respect to images of the portion of the patient anatomy. The images of the portion of the patient anatomy may include, for example, one or more 2D images derived from intra-operative 3D images (e.g., a 3D CT scan volume) or a 3D model created via segmentation from the intra-operative 3D images (e.g., a 3D CT scan volume).

In accordance with several embodiments, the above registration and display approach requires a 3D intra-operative scanner (e.g., O-arm scanners) in the operating room. The images from this type of scanner may be of a lower quality (e.g., lower image resolution, increased noise artifacts) compared to a pre-operative scanner (e.g., pre-operative CT or MRI or PET scanner or other imaging device, such as an ultrasound imaging device). Pre-operative CT scanners may include, for example, spiral CT scanners, dual energy CT scanners, multi-slice or multi-scan CT scanners, cone beam CT scanners, or photon-counting CT scanners.

One way to provide image-guided medical procedure navigation (e.g., augmented reality-assisted spinal navigation) without requiring a 3D intra-operative scanner is to register the patient using an intra-operative 2D scanner (e.g., X-ray fluoroscopy imaging machine, such as a C-arm fluoroscopy machine) and then matching a pre-operative 3D scan (e.g., 3D CT or MRI) to the registered 2D images (e.g., X-rays). This matching may be performed globally (e.g., in which a best registration of the entire pre-operative 3D imaging is made relative to the 2D imaging) or locally. For local matching, the pre-operative 3D scan (e.g., CT scan) may be segmented into separate individual rigid bodies (e.g., individual vertebrae of a spine) and each rigid body may then be registered to corresponding rigid bodies using the 2D imaging. Once the pre-operative 3D scan (e.g., CT scan) is registered, navigation and visualization may then be performed as before, but the imagery and the 3D models are now derived from a high quality (higher resolution, lower noise, etc.) pre-operative imaging (e.g., CT scan). This results in improved visualization quality to provide an enhanced user experience and satisfaction. Examples of CT-Fluoro registration and local matching implementations are described in International PCT Publication No. WO 2024/018368.

In accordance with several embodiments, systems, devices and methods are described that provide improved quality of augmented reality displays for medical professionals to visualize as they perform image-guided medical procedure navigation (e.g., medical treatment, diagnostic, and/or other intervention procedures) even when registration is based on intra-operative 3D scans (e.g., intra-operative CT scans) and not on 2D fluoroscopy imaging. For example, the systems, devices and methods disclosed herein may advantageously facilitate augmented-reality assisted medical procedure navigation using displays based on pre-operative three-dimensional (3D) imaging or images (e.g., pre-operative tomographic imaging, such as CT scans, or MRI) of at least a portion of patient anatomy (e.g., portion of a spine or other bone, joint or soft tissue) instead of using displays based on intra-operative imaging or images, which may be of a lower quality (e.g., lower image resolution, higher noise artifacts) than the pre-operative images.

A computing system including one or more processors that execute program instructions stored in memory may register the patient using a lower image quality intra-operative 3D scan (e.g., cone-beam CT scan) and then match this registration to (e.g., determine a transformation between the intra-operative imaging and) a higher image quality pre-operative 3D scan, thereby obtaining the benefit of high-quality visualization to facilitate the image-guided medical procedure navigation. The registration to the patient using the intra-operative 3D scan may involve use of a registration marker that includes first elements (e.g., radiopaque elements) visible to the intra-operative 3D imaging modality and second elements (e.g., optically-reflective elements) visible to an optical imaging modality employed by an optical imaging device (e.g., infrared camera or sensor), which may be positioned on a wearable head-mounted display device (or a separate tracking device in alternative implementations). The registration to the patient may also involve identification of a location of a patient marker coupled directly or indirectly to a portion of patient anatomy corresponding to the medical procedure that includes optically-reflective elements that are also visible to the optical imaging device. The locations and orientations of the registration marker and the patient marker with respect to each other can then be determined.

The registration matching process may be performed globally (e.g., in which a best registration of the entire pre-operative 3D imaging is made relative to the intra-operative 3D scan) or locally. In the local approach, the pre-operative 3D scan (e.g., CT scan or MRI scan) may be segmented into rigid bodies and a 3D-3D match process may be performed (e.g., using automated or semi-automated feature matching processes or algorithms) to determine the transformation between the pre-operative segment and the corresponding intra-operative segment in order to properly align the pre-operative segment into the current position determined intra-operatively using the intra-operative scan registered to the fiducials (e.g., one or more markers) attached to anatomy of a patient or otherwise placed on the patient.

The medical procedures may include spinal surgery procedures, other orthopedic procedures (such as procedures involving the hip, knee, ankle, elbow, shoulder, foot, arm, leg), cranial procedures, dental or oral surgery procedures, ear-nose-throat (ENT) procedures, or other procedures. The systems and methods described herein may be used in connection with surgical procedures, such as spinal surgery, joint surgery (e.g., shoulder, knee, hip, ankle, other joints), orthopedic surgery, heart surgery, bariatric surgery, facial bone surgery, dental surgery, cranial surgery, or neurosurgery. The surgical procedures may be performed during open surgery or minimally-invasive surgery (e.g., surgery during which small incisions are made that are self-sealing or sealed with surgical adhesive or minor suturing or stitching). The systems and methods described may also be used in connection with other medical procedures (including therapeutic and diagnostic procedures) and with other instruments and devices or other non-medical display environments. The methods described herein further include the performance of the medical procedures (including but not limited to performing a surgical intervention such as treating a spine, shoulder, hip, knee, ankle, other joint, jaw, cranium, etc.).

In accordance with several embodiments, the systems, devices, and methods described herein may facilitate acquiring and processing 3D images (e.g., CT scan, MRI, ultrasound, etc.) of a patient's spine or other anatomical region prior to a surgical or other medical treatment or diagnostic procedure (e.g., hours or days or weeks or months prior), and using them in combination with 3D intra-operative images of the patient taken during the procedure (e.g., minutes or seconds prior to actual performance of a medical intervention or during the actual performance of the medical intervention). The software in the system may include algorithms that segment the pre-operative 3D scans (e.g., CT scans or MRI scans) of an anatomical region (e.g., at least a portion of a spine) into individual anatomical components (e.g., individual vertebrae, sacrum, and ilium for the spine and pelvic region).

Also contemplated herein are methods and computer software products for performing functions of any preceding implementation or apparatus.

Also contemplated herein are apparatus and computer software products for performing any preceding implementation.

Also contemplated herein are the use of the apparatus, systems, or methods of any preceding implementation to perform calibration and registration between pre-operative 3D images and intra-operative 3D images.

Also contemplated herein are the use of any of the apparatus, systems, or methods of any of the preceding implementations for the treatment of a spine through surgical intervention.

Also contemplated herein is the use of any of the apparatus, systems, or methods for the treatment of a spine through a surgical intervention.

Also contemplated herein is the use of any of the apparatus, systems, or methods for the treatment of an orthopedic joint through a surgical intervention, including, optionally, a shoulder, a knee, an ankle, a hip, or other joint.

Also contemplated herein is the use of any of the apparatus, systems, or methods for the treatment of a cranium through a surgical intervention.

Also contemplated herein is the use of any of the apparatus, systems, or methods for the treatment of a jaw through a surgical intervention.

Also contemplated herein is the use of any of the apparatus, systems, or methods for diagnosis of a spinal abnormality or degeneration or deformity.

Also contemplated herein is the use of any of the apparatus, systems, or methods for diagnosis of a spinal injury.

Also contemplated herein is the use of any of the apparatus, systems, or methods for diagnosis of joint damage.

Also contemplated herein is the use of any of the apparatus, systems, or methods for diagnosis of an orthopedic injury.

In accordance with several embodiments, any of the methods described herein may include diagnosing and/or treating a medical condition, the medical condition comprising one or more of the following: back pain, spinal deformity, spinal stenosis, disc herniation, joint inflammation, joint damage, ligament or tendon ruptures or tears.

In accordance with several embodiments, a method of presenting one or more images on a wearable display is described and/or illustrated herein during medical procedures, such as orthopedic procedures, spinal surgical procedures, joint repair procedures, joint replacement procedures, facial bone repair or reconstruction procedures, ENT procedures, cranial procedures or neurosurgical procedures.

For purposes of summarizing the disclosure, certain aspects, advantages, and novel features of embodiments of the disclosure have been described herein. It is to be understood that not necessarily all such advantages may be achieved in accordance with any particular embodiment of the disclosure disclosed herein. Thus, the embodiments disclosed herein may be embodied or carried out in a manner that achieves or optimizes one advantage or group of advantages as taught or suggested herein without necessarily achieving other advantages as may be taught or suggested herein. The systems and methods of the disclosure each have several innovative aspects, no single one of which is solely responsible for the desirable attributes disclosed herein. The methods summarized above and set forth in further detail below describe certain actions taken by a practitioner; however, it should be understood that they can also include the instruction of those actions by another party. Thus, actions such as “obtaining one or more pre-operative 3D images” include “instructing the obtaining of one or more pre-operative 3D images.”

The disclosure will be more fully understood from the following detailed description of the embodiments thereof, taken together with the drawings.

### BRIEF DESCRIPTION OF THE DRAWINGS

The following drawings are for illustrative purposes only and show non-limiting embodiments. Features from different figures may be combined in several embodiments. It should be understood that the figures are not necessarily drawn to scale. Distances, angles, etc. are merely illustrative and do not necessarily bear an exact relationship to actual dimensions and layout of the devices illustrated.

FIG. 1 is a schematic pictorial illustration of a system for facilitating augmented reality image-guided procedures performed by one or more clinical professionals, in accordance with an embodiment of the disclosure.

FIG. 2A is a schematic pictorial illustration of a head-mounted unit for use in the system of FIG. 1.

FIG. 2B is a schematic pictorial illustration of another head-mounted unit for use in the system of FIG. 1.

FIG. 3 is a flowchart of an augmented-reality image visualization workflow that involves registration and transformation using pre-operative and intra-operative 3D imaging.

FIG. 4 is a flowchart of an embodiment of a process to facilitate high-quality augmented reality images to facilitate navigation and visualization during medical procedures.

### DETAILED DESCRIPTION

Embodiments of the disclosure that are described hereinbelow provide apparatuses, methods, and software for image registration and display, particularly for facilitating image-guided, augmented reality-assisted navigation during medical treatment and/or diagnostic procedures (e.g., open surgery or minimally invasive surgery, such as laparoscopic surgery or endoscopic surgery). Embodiments of the disclosure that are described hereinbelow may include systems, devices, methods, and software for segmentation of medical images for various purposes.

In some systems for image-guided surgery or other medical intervention, anatomical images of structures inside the patient's body are overlaid on the clinical professional's actual view of the patient's body, generating an augmented reality view that can be used to facilitate visualization of patient anatomy during a minimally-invasive medical procedure and navigation by a viewer of the augmented reality view (e.g., a wearer of a head-mounted AR display device). Display of 3D anatomical images in this manner, such as CT or magnetic resonance (MR) images or virtual models, meshes, or images generated therefrom, can be especially useful in enabling the surgeon or other clinician to visualize structures that are hidden from actual view by overlying layers of tissue or bone. During orthopedic surgery, for example, the augmented reality (AR) display may show 3D images or models of bone segments overlaid on (to appear to the wearer as being aligned with) the locations of the corresponding bones in a target region of the patient's body. For example, during spinal surgery, 3D images of the vertebrae may be or appear to be overlaid on the skin of the patient's back for minimally invasive surgery or overlaid on the actual vertebrae for open surgery. The display may additionally or alternatively include display of 2D images (e.g., X-ray images or digitally reconstructed radiograph images derived from 3D images, such as CT images) to facilitate AR navigation.

In image-guided surgery or other medical intervention, it can be important that the anatomical images displayed to the surgeon to provide guidance and/or facilitate navigation (e.g., of medical tools and instruments) within the patient body, correspond to the current anatomy of the patient (e.g., pose and/or structure). In addition, for this sort of AR display or view to be clinically useful, it can be important that the overlaid anatomical images (or segmented models generated therefrom) be properly registered with the actual anatomical structures in the body. When intra-operative images are acquired during the operation or procedure, for example using an intra-operative medical imaging scanner, such as a CT or MRI scanner, proper registration will be maintained as long as the patient is stationary.

In most surgeries or other medical interventions, pre-operative images (e.g., 3D images or volumes stored as DICOM data) are acquired before the surgery or other medical intervention, in a different room, and, for example, the patient's pose on the operating table is often different from that in the pre-operative images (e.g., tomographic images, ultrasound images, or MR images). 3D images or volumes acquired pre-operatively typically will not have a reference (e.g., a fiducial marker) that will allow the registration of the pre-operative 3D images with the patient anatomy at the time of the operation. In addition, there may be a change in the relative location of individual anatomical components (e.g., vertebrae in the patient's spine) between the time the pre-operative 3D images were acquired and the time of the operation or other medical intervention. Such a change may be due to a change in the patient's pose, an insertion of an implant, or any other reason.

Intra-operative images obtained during the surgery or other medical intervention may be of a lower quality (e.g., lower image resolution, higher noise artifacts, etc.) than the pre-operative images. Accordingly, if the intra-operative images are used to generate the 3D images or models that are generated for display as an augmented reality image to a wearer of a head-mounted display device, the displayed images may be of a lower quality than images or models that are generated for display using the pre-operative images, which may have a higher image resolution, fewer noise artifacts, etc., than the intra-operative images). The improved augmented reality images may provide an improved experience for the wearer (e.g., surgeon or clinical professional), which may result in increased adoption of the technology and result in more streamlined, safe, and efficient procedures.

Embodiments of the disclosure that are described herein provide methods, systems, and computer software products that can be used to register pre-operative 3D medical imaging (e.g., one or more CT images or MR images) with intra-operative 3D scan imaging (e.g., intra-operative CT scan imaging). In some of the disclosed methods, systems and computer software products, the pre-operative 3D imaging is segmented into multiple 3D segments, for example, each containing a respective one of the vertebrae for spinal implementations. For spinal implementations, each of these 3D segments may be registered (e.g., via a local 3D-3D matching process, such as using feature matching algorithms) with a respective vertebra in the intra-operative images. Specifically, the respective location and orientation of each 3D segment may be adjusted to match the respective vertebra in the intra-operative images and thus to account for changes in the relative location of vertebrae (e.g., due to a change in the patient's pose on the operating table relative to the pose in the pre-operative 3D image). The matching may involve comparison of image similarity values (e.g., pixel value comparison), for example.

In some implementations, the intra-operative 3D imaging may also be segmented into multiple 3D segments.

Similar techniques may be used for other types of surgery or medical interventions. For example, in some implementations, the pre-operative 3D image may be segmented into other bony portions or components.

In the context of spine surgery, when the registration process is complete, an image of the spine comprising the registered 3D segments (e.g., a 3D model) from the pre-operative 3D imaging is presented on a display, for example by overlaying an AR image of the 3D model of registered 3D segments on the back (or at least causing the AR image to appear to the wearer as if it is overlaid on the back aligned with the actual patient anatomy underneath the skin) of the patient to generate an AR view. In some embodiments, the actual native 3D images (e.g., direct volume rendered images) are not displayed.

Similar techniques may be employed for non-spinal implementations and the calibration and registration and display may be tailored to the specific anatomy relevant to a particular medical intervention (e.g., other orthopedic surgery or intervention, cranial surgery or other intervention, ENT surgery or other intervention, oral surgery or other intervention). Similar techniques may also be employed using only 2D images (e.g., one or more X-rays) without relying on 3D images.

A processor (e.g., one or more processing devices or units) may be configured to receive images captured in the operating room, including one, two, or more intra-operative 3D images captured by an intra-operative 3D scanner or imaging device (which contain an image of a registration marker that includes elements that are visible to the 3D imaging modality). The processor (e.g., one or more processing devices or units) also receives one or more images from an optical imaging device (e.g., camera or sensor operating in the infrared and/or visible wavelength range) that also captures position and orientation of elements of the registration marker that are visible to the optical imaging device (e.g., reflective elements that are aligned with radiopaque elements visible to the 3D imaging modality, such as a CT scanner). The optical imaging device may be located on the wearable head-mounted display unit.

In some embodiments, the processor (e.g., one or more processing devices or units) processes the intra-operative image(s) together with the optical image(s) acquired by the optical imaging device so as to calibrate and register the pre-operative images with the body of the patient. For this purpose, the processor (e.g., one or more processing devices or units) may compute a registration between the intra-operative imaging and the body of the patient using one or more registration markers and one or more patient markers (as well as an optical imaging device of a head-mounted display unit), such as described in PCT Publication No. WO 2019/211741, which is incorporated herein by reference in its entirety. Then a registration or transformation between the pre-operative imaging and the body of the patient is determined based on matching between corresponding identified structures of the intra-operative imaging and the pre-operative imaging, thereby facilitating augmented reality display of the higher quality pre-operative imaging (and/or new 3D volumes or models generated from the pre-operative imaging) with the body of the patient using the display of the head-mounted display unit.

In some embodiments, the processor (e.g., one or more processing devices or units) applies the registration and transformation in presenting an image (3D model) of anatomical structures (e.g., individualized vertebrae, a portion of a spine (lumbar, sacral, lumbosacral, cervical, thoracic), a whole spine, pelvic bones, leg bones, arm bones, hip bones, knee joints, ankle or foot bones, hand bones, brain tissue, cranial bones, oral and maxillofacial bones, bone joints such as sacroiliac joints, organs or other soft tissue, etc.) in the body of the patient on a display, such as an AR display. Additionally or alternatively, other sorts of information may be integrated into the AR image. For example, the display may incorporate information from a pre-acquired 3D tomographic image, such as a CT image or an MRI image, or other medical image. For this purpose, it is desirable that the tomographic or other medical image also be registered with the body of the patient.

In accordance with several embodiments, it may be desired to combine image information from multiple different imaging modalities and present the combined image information to the surgeon or other clinical professional, for example on an AR display of a wearable device, such as a head-mounted unit or eyewear (e.g., goggles, visor, or glasses) and/or on a non-wearable device, such as a tablet, portal monitor, or workstation display. Each imaging modality and device may have its own frame of reference, which is separate and independent from the other modalities and devices, and is typically subject to distortions of different types. These imaging modalities may include, for example, optical cameras that are used to capture visible and/or infrared images of the patient's body; a fluoroscope, which captures 2D X-ray images of the patient's body in the operating room or diagnostic room; and medical imaging scanners (e.g., tomographic scanners, such as CT scanners and MRI scanners, which may be used to capture preoperative or intra-operative 3D scans of the body. Ultrasound scanners or other 3D or 2D imaging modalities may also be used. In some aspects, the imaging modalities must be capable of imaging bone tissue.

In accordance with several embodiments, to combine image information from such different sources in a way that can give useful guidance to the surgeon or other clinical professional, it is desirable that all the imaging frames of references be calibrated and registered with the frame of reference of the patient's body (and thus all the imaging frames are registered with one another). For certain embodiments of AR displays, in which image information from these sources is visually overlaid on the body itself, precise calibration and registration can be critical to facilitate accurate and precise navigation by the surgeon or other clinical professional relying on the AR display.

The terms “image” and “images,” as will be used hereinafter, may include 2D images and/or 3D images, including computer-generated 2D or 3D renderings or models, such as digitally reconstructed radiographs (DRRs).

Several embodiments are particularly advantageous because they include one, several, or all of the following benefits: (i) improved visualization of structures within the body during surgery; (ii) enhanced accuracy in planning and carrying out surgical procedures; (iii) increased availability of AR-assisted navigation without sacrificing accuracy or precision; and/or (iv) enrichment of the surgeon's understanding of 3D features of the patient's anatomy.

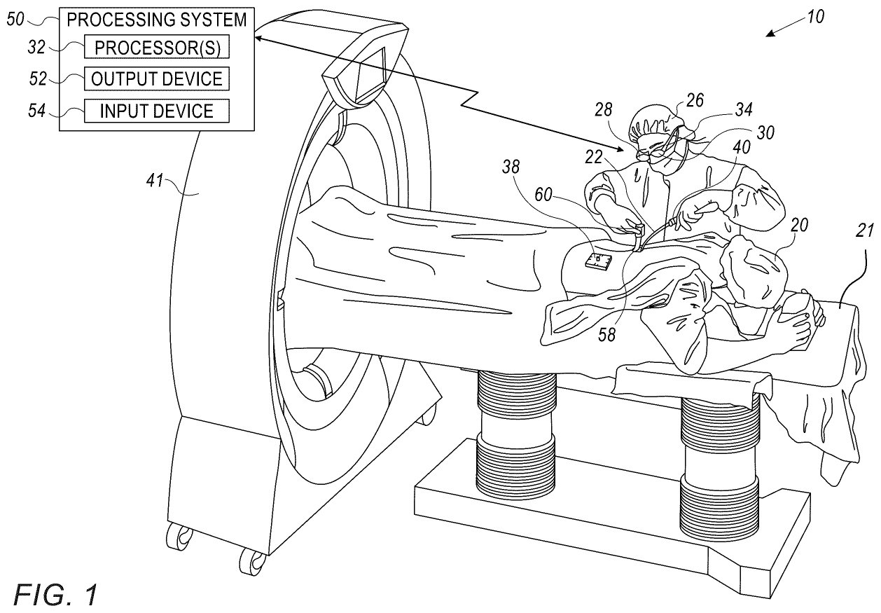

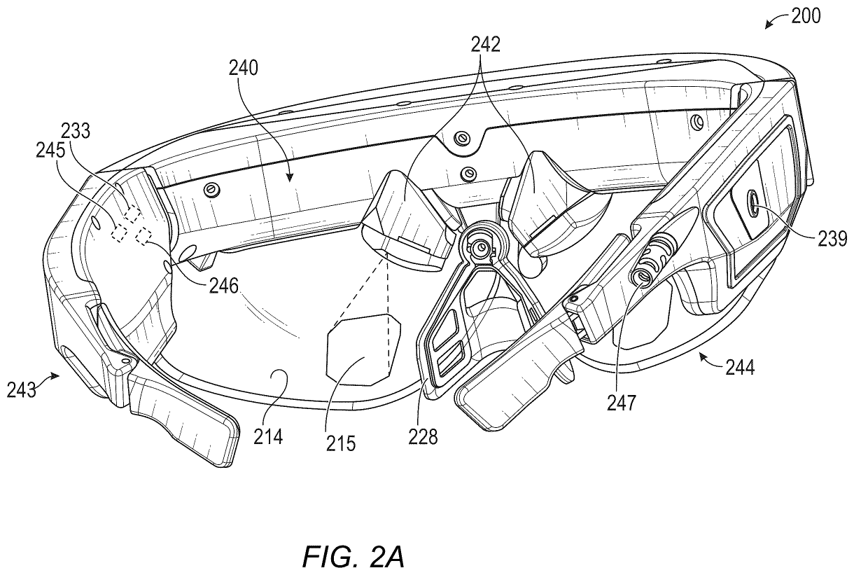

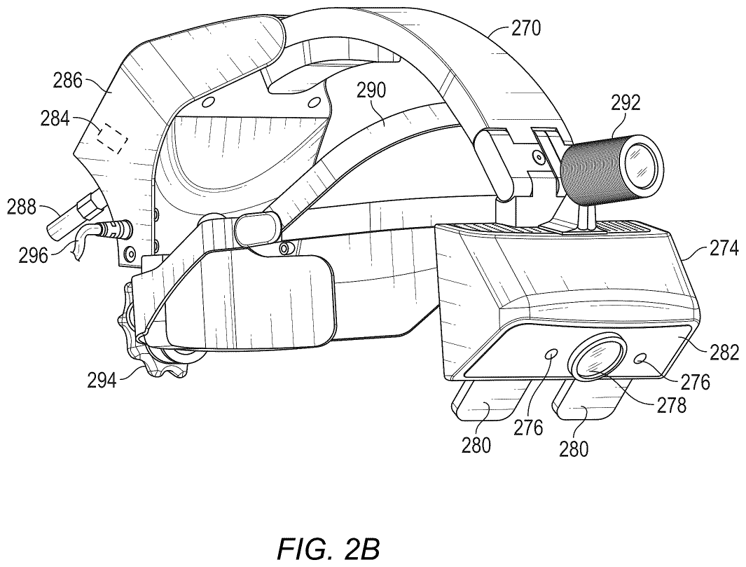

FIG. 1 is a schematic pictorial illustration of a system 10 for image-guided surgery or other medical intervention using AR-assisted navigation, in accordance with an embodiment of the disclosure. In the pictured scenario, a surgeon or other clinical professional 26 is preparing to operate on the spine of a patient 20, who is lying on an operating table 21. The surgeon or other clinical professional 26 views the patient's back through a head-mounted AR display unit 28, examples of which are shown in greater detail in FIGS. 2A and 2B. The AR display unit may be in the form of a head-mounted unit, such as eye-glasses, spectacles, other eyewear, a helmet, a visor, or other over-the-head mounted units. Alternatively, other wearable or hands-free devices, units, or displays may be used.

In accordance with several embodiments, system 10 is applied in a medical procedure on a patient 20 using image-guided intervention. In some procedures, a tool 22 is inserted via an incision (e.g., minimally-invasive or self-sealing incision or during open surgery) in the patient's back in order to perform a surgical intervention. Alternatively, system 10 and the techniques described herein may be used, mutatis mutandis, in other surgical or non-surgical medical or diagnostic procedures.

In the pictured embodiment, a user of system 10, such as a healthcare professional 26 (e.g., a surgeon performing the procedure), wears the head-mounted display unit 28. In various embodiments, the head-mounted display unit 28 includes one or more see-through displays 30, for example as described in the above-mentioned U.S. Pat. No. 9,928,629 or in the other patents and applications cited above that are incorporated by reference. Such displays may include an optical combiner that is controlled by one or more computer processors (e.g., one or more computer processors 32 in a central processing system 50 and/or one or more dedicated computer processors in the head-mounted display unit 28) to display an augmented-reality image to the healthcare professional 26. The image is presented on displays 30 (e.g., as a stereoscopic near-eye display) such that a computer-generated image is projected in alignment with the anatomy of the body of patient 20 that is visible to professional 26 through a portion of the display.

In some embodiments, one or more projected computer-generated images include a virtual image of tool 22 overlaid on a virtual image (e.g., 3D model) of at least a portion of the patient's anatomy (such as slices, a 3D model or 2D images derived from the 3D medical image, e.g., of a portion of the spine). Specifically, a portion of the tool 22 that would not otherwise be visible to the healthcare professional 26 (for example, by virtue of being hidden by the patient's anatomy) is included in the computer-generated image.

In image-guided surgery or in surgeries utilizing augmented reality systems, a bone-anchoring device may be used as a fiducial marker or may be coupled with such a marker, for indicating the patient's body location in a coordinate system. In system 10, for example, an anchoring device 58 is coupled with a marker 60 that is used to register a region of interest (ROI) of the patient body with medical images of the ROI (e.g., preoperative and/or intra-operative medical images that include at least the ROI). During the procedure, a tracking system (e.g., an infrared (IR) tracking system of the head-mounted display unit 28) tracks the marker 60 mounted on the anchoring device 58 and the tool 22 that includes a tool marker 40. Following that, the display of the medical image data (which may include, for example, a virtual 3D model generated based on such actual patient anatomical data) on a near-eye display may be aligned with the professional's actual view of the ROI based on this registration. In addition, a virtual image of the tool 22 may be displayed on the virtual 3D model or other medical image based on the tracking data and the registration. The user (e.g., professional 26) may then navigate the tool 22 based on the virtual display of the tool 22 with respect to the patient image data, and optionally, while the 3D model is aligned with the professional's view of the patient or ROI.

According to some aspects, the 3D image or model presented on display 30 is aligned with the patient's body. According to some aspects, allowed alignment error (or allowed misalignment) may not be more than about two to three millimeters (mm) or may be less than four mm or less than five mm or about one to two mm or less than one mm. In one embodiment, the allowed alignment error is less than one mm. In order to account for such a limit on error in alignment of the patient's anatomy with the presented images, the position of the patient's body, or a portion thereof, with respect to the head-mounted unit may be tracked. According to some aspects, the 3D model presented on display 30 is misaligned with the patient's body (e.g., having a misalignment greater than the allowed misalignment).

A patient marker 60 attached to the anchoring implement or device 58 (such as a clamp or a pin, for example) may be used for this purpose, as described further hereinbelow. In some embodiments, a registration marker, such as registration marker 38, may be used for registering the 3D image with the patient anatomy, e.g., in a preceding registration procedure. In some embodiments registration marker 38 may be removed once the registration is complete. Anchoring devices, markers and registration systems and methods of this sort for image-guided surgery are described, for example, in the above incorporated U.S. Pat. Nos. 9,928,629, 10,835,296 and 10,939,977, and in addition in Applicant's U.S. Patent Application Publication 2021/0161614, U.S. Patent Application Publication 2022/0142730, U.S. Patent Application Publication 2021/0386482, U.S. Patent Application Publication 2023/0009793, and PCT International Publication WO 2023/026229, all of which are incorporated herein by reference in their entirety.

When an image of tool 22 is incorporated into the image that is displayed on head-mounted display unit 28, the position of the tool 22 with respect to the patient's anatomy should be accurately reflected. For this purpose, the position of the tool 22 or a portion thereof (e.g., a tool marker 40) is tracked by system 10 (e.g., via a tracking system built into the head-mounted display unit 28). In some embodiments, it is desirable to determine the location of the tool 22 with respect to the patient's body such that errors in the determined location of the tool 22 with respect to the patient's body are typically less than three mm or less than two and a half mm or less than two mm.

In some embodiments, head-mounted display unit 28 includes a tracking sensor 34 to facilitate determination of the location and orientation of head-mounted display unit 28 with respect to the patient's body (e.g., via patient marker 60) and/or with respect to tool 22 (e.g., via a tool marker 40) . Tracking sensor 34 can also be used in finding the position and orientation of tool 22 (e.g., via tool marker 40) with respect to the patient's body (e.g., via patient marker 60). In one embodiment, tracking sensor 34 comprises one or more image-capturing or image-acquiring devices, such as a camera (e.g., infrared camera, visible light camera, and/or an RGB-IR camera), which captures or acquires images of patient marker 60 and/or tool marker 40, and/or other landmarks or markers. The tracking sensor 34 may comprise an optical tracking device, an RFID reader, an NFC reader, a Bluetooth sensor, an electromagnetic tracking device, an ultrasound tracking device, or other tracking device.

In the pictured embodiment, system 10 also includes a tomographic imaging device, such as an intra-operative CT scanner 41. Alternatively or additionally, processing system 50 may access or otherwise receive tomographic data from other intra-operative imaging sources (e.g., an MRI scanner, ultrasound imaging device, or a PET imaging device); and the CT scanner 41 itself is not necessarily an essential part of the present system 10 (e.g., it is optional). Regardless of the source of the intra-operative imaging data, the one or more processors 32 compute a transformation over the ROI so as to register the intra-operative images with images and information that are displayed on head-mounted display unit 28 based on pre-operative imaging. The processor(s) 32 can then apply this transformation to pre-operative imaging by performing 3D-3D matching algorithms between corresponding portions or features of the intra-operative imaging and the pre-operative imaging to allow for display of a 3D model based on the pre-operative imaging on display 30 in registration with the ROI viewed through the display 30. In the disclosed technique, the processor 32 can generate from the pre-operative images a 3D model, and register the 3D model with the ROI, e.g., viewed through the display 30.

In accordance with several implementations, in order to generate and present an augmented reality image on display 30, processor 32 computes the location and orientation of head-mounted display unit 28 with respect to a portion of the body of patient 20 (e.g., the patient's back or a portion thereof). In some embodiments, processor(s) 32 also compute the location and orientation of tool 22 with respect to the patient's body. In one embodiment, one or more processors that are integrated within the head-mounted display unit 28, may perform these functions or a portion of these functions. Alternatively or additionally, processor(s) 32, which are disposed externally to the head-mounted display unit 28 and which may be in wireless communication with the head-mounted display unit 28, may be used to perform these functions or a portion of these functions. Processor(s) 32 can be part of processing system 50, which additionally includes an output device 52 (e.g., a display, such as a monitor) for outputting information to an operator of the system, memory, and/or an input device 54 (such as a pointing device, a keyboard, a mouse, a trackpad, a pedal or touchscreen display, etc.) to allow the operator (e.g., professional 26 or an assistant to professional 26) to input data into the system 10.

In general, in the context of the present description, when a computer processor is described as performing certain steps, these steps may be performed by external computer processor(s) 32 and/or one or more computer processor(s) that are integrated within the head-mounted display unit 28. The processors described herein (e.g., processor 32) may include a single processor or multiple processors (e.g., parallel processors running in parallel to reduce computing time). If multiple processors are used, they may be communicatively coupled to each other over a network (e.g., wired or wireless communications network). The processor or processors carry out the described functionality under the control of suitable software, which may be downloaded to system 10 in electronic form, for example over a network, and/or stored on tangible, non-transitory computer-readable media, such as electronic, magnetic, or optical memory. In some embodiments, processing system 50 may be integrated in head-mounted display unit 28.

In some embodiments, system 10 may include a see-through and/or augmented reality display which is not head-mounted. In such systems the display may be mounted to patient 20 or to the operating table and positioned such that professional 26 may be able to view patient 20 anatomy and/or operation site through at least a portion of the display. The display position may be adjustable.

In some embodiments, the 3D image, 3D rendering and/or 3D model of the present disclosure may be displayed while allowing user interaction as described herein on a display which is not a see-through display and/or a head-mounted display, such as a display of a workstation, a personal computer, a terminal, a tablet or a mobile device.

The head-mounted display unit 28 may refer to a helmet, AR glasses, goggles, spectacles, monocle, eyewear, headset, visor, heads-up display (HUD), and any other suitable type of displaying device mounted on or worn by any portion of a user or wearer's head, including but not limited to the face, crown, forehead, nose, and ears.

In some embodiments, the head-mounted display unit 28 is used together with stand-alone displays, such as monitors, portable devices, tablets, etc. Displays 30 may be controlled by processor 32 (e.g., by a processor unit of processor 32 disposed on head-mounted AR display unit 28, not shown in the figures) to display an AR image to surgeon 26, who is wearing the head-mounted AR display unit 28.

The head-mounted display unit 28 may be substituted with an alternative hands-free device that is not worn by the operator, such as a portal, monitor, or tablet.

In some embodiments, surgeon or other medical professional 26 may attach a patient marker 60 to a bone in the body of patient 20, for example to the patient's spine, using a suitable clamp (e.g., spinous process clamp) or pin (e.g., iliac pin). A marker of this sort is described, for example, in U.S. Pat. No. 10,939,977, whose disclosure is incorporated herein by reference in its entirety. Additionally or alternatively, surgeon or other medical professional 26 may fix a registration marker 38 to the patient's body surface, for example as shown in FIG. 1. A registration procedure utilizing a registration marker attached or adhered to (directly or indirectly) the patient's back, is described, for example, in U.S. Patent Application Publication 2021/0161614, which disclosure was previously incorporated herein by reference. Alternatively, surgeon or other medical professional 26 may use a registration marker mounted on the patient's spine via a supporting or mounting structure such as a clamp or a pin. A registration procedure utilizing a registration marker mounted on a patient's spine via such a supporting or mounting structure is disclosed, for example, in U.S. Patent Application Publication 2022/0142730, which disclosure is likewise hereby incorporated herein by reference in its entirety. In some implementations, the tracking sensor 34 (e.g., infrared camera or other optical camera) captures images including both registration marker 38 and/or patient marker 60 attached to patient 24. Although tracking sensor 34 in FIG. 1 is mounted on head-mounted AR display unit 28, these images may alternatively be captured by one or more suitable optical cameras (e.g., infrared camera) mounted elsewhere on the head or body of surgeon or other medical professional 26 or mounted elsewhere in the operating room (e.g., in a stationary manner).

In addition, processing system 50 may also receive pre-operative 3D tomographic or other medical images of patient 24 (e.g., CT or MRI images), and store these pre-operative 3D images in a memory 52. For spinal interventional procedures, processing system 50 may segment the pre-operative 3D images and match the 3D segments with respective vertebrae in intra-operative 3D images. The processing system 50 may then present an image (e.g., a 3D model) of the spine comprising the registered pre-operative 3D segments on head-mounted AR display unit 28, such that the vertebrae in the 3D images are aligned with the actual vertebrae of the patient's spine. Such presentation may facilitate AR-assisted navigation during a surgical procedure or other medical intervention (e.g., therapeutic and/or diagnostic intervention). Details of this process are described herein. Similar processes may be performed for other joints, bones, or tissue (e.g., cartilage, nervous tissue, or other soft tissue).

Alternatively or additionally, processing system 50 may present image information on a different sort of display, for example on an AR display that is mounted on patient 20 or on operating table 21 above the surgical site or at another location within the operating room, such as a stationary display (e.g., a workstation display) located in the operating room.

Processing system 50 may comprise one or more general-purpose computer processors, which is or are programmed in software (via computer-readable program instructions) to carry out the functions of segmentation, calibration, registration, and/or display that are described herein. This software may be stored on tangible, non-transitory computer-readable media, such as optical, magnetic, or electronic memory media. Additionally or alternatively, at least some of the functions of processing system 50 may be carried out using special-purpose computing hardware, such as a graphics processing unit (GPU), which may include, for example, multiple units.

FIG. 2A is a schematic pictorial illustration showing details of head-mounted AR display unit 200, in accordance with an embodiment that can be used as the head-mounted display unit 28 in FIG. 1. Head-mounted display unit 200 is in the form or substantially in the form of glasses, spectacles, goggles, or other eyewear. Head-mounted display unit 200 includes see-through displays, for example as described in the above-mentioned U.S. Pat. No. 9,928,629 or PCT International Publication WO 2022/053923. The see-through displays may comprise optical see-through displays, video see-through displays, or a hybrid combination of both. The see-through displays may comprise a stereoscopic display. In some implementation, the AR image(s) are projected onto an overlay area of displays in alignment with the anatomy of the body of patient 20, which is visible to surgeon or other health care professional 26 through displays. The AR image(s) may include, for example, anatomical features, such as images or 3D models or representations of bones taken from tomographic or volumetric images and/or graphical representations of tools inside the patient's body, as well as surgical guidance and planning data or other information. The AR image(s) may be overlaid on the actual locations of the anatomical features of patient 20 that are viewed by surgeon or other health care professional 26. In some implementations, the AR image(s) are presented directly into or onto the retina of one or both of the patient's eyes.

To align the AR image with the patient's anatomy, one or more cameras (e.g., infrared or other optical cameras) may be configured to capture respective images of a field of view (FOV), which includes one or more of markers 38, 40, 60 for registration purposes. In some embodiments, processor(s) 32 process the images of one or more of the markers to register the location and orientation of the head-mounted display unit 200 with the patient's body. Based on this registration, processor(s) 32 are able to select the appropriate features to display in the AR image in the overlay area (which may be displayed directly on a wearer's retina) and to set the appropriate magnification, translation, and orientation to match the underlying structure of the patient's anatomy as seen from the point of view of surgeon 22 or other clinical professional.

In some embodiments, the head-mounted display unit 200 comprises two optical engines (OEs) 240, one for each eye. In some embodiments, each OE 240 comprises an AR projector 242, which is configured to direct the AR image described in FIG. 1 above, to a respective AR display 215. In one example, each OE 240 comprises an OLED-based image source and display configured to project the AR image produced by processor(s) 32, and optics (e.g., one or more lenses and mirrors) configured to direct the AR image to AR display 215.

In some embodiments, AR display 215 is a section of visor 214, which is coated with one or more suitable layers, which is or are configured to reflect the projected VR image to the pupil of the respective eye, so that surgeon 26 can see the VR image overlaid on a scene of interest (e.g., the organ (or other target treatment anatomy or region) being operated on), in a way of augmented vision, virtual overlay on the real world.

In some embodiments, visor 214 is fully transparent or partially transparent so that when directing the gaze away from AR display 215, surgeon 26 can see the scene around him or her without having the AR image overlaid thereon.

In some embodiments, head-mounted display unit 200 comprises two temple arms described herein, and nose pad 228 for mechanically supporting the mounting of head-mounted display unit 200 over the head of surgeon 26.

In some embodiments, a left temple arm 243 comprises processor 233 and optionally other devices, such as a wireless communication device 245 configured to exchange signals between head-mounted display unit 200 and external entities, and a storage device 246 configured to store images, signals, program instructions and additional data of head-mounted display unit 200. Note that processor 233, wireless communication device 245, and storage device 246 appear in dashed lines for being embedded within the inner volume of left temple arm 243.

In some embodiments, processor 233, wireless communication device 245, and storage device 246 may be disposed on one or more suitable substrates, such as one or more printed circuit boards (PCBs).

In some embodiments, all the devices are disposed on a single rigid PCB. In some embodiments, at least one of the PCBs may be flexible.

In some embodiments, head-mounted display unit 200 comprises a right temple arm 244, which comprises an on/off or standby button 239 configured to turn the power on when using HMD 22 and to turn the power off when HMD 22 is not in use.

In some embodiments, temple arms 243 and 244 are configured to be adjusted to the shape of the respective left and right temples of surgeon 26 or of any other user, and to be mounted on the ears of surgeon 26 in order to hold the weight of head-mounted display unit 200 (in one example, together with a nose pad described below).

In some embodiments, nose pad 228 is configured to be adjusted to the shape of the nose of surgeon 26 or to the nose of any other user.

In the context of the present disclosure, the terms “frame” and “head mounting assembly” are used interchangeably and may refer to the combination of two or more elements among a housing, nose pad 228 and temple arms 243 and 244, or any other suitable assembly configured to hold head-mounted display unit 200 at a selected position on the head of surgeon 26.

In some embodiments, a power cable (not shown) is threaded through a power cable strain relief 247 of head-mounted display unit 200. In the present configuration, power cable strain relief 247 is mounted on right temple arm 244, and the power cable is configured to electrically connect between a power source (not shown) and several components of head-mounted display unit 200, such as but not limited to an on/off button 239.

In some embodiments, the power source comprises a pack of suitable batteries, and one or more supercapacitors or ultracapacitors (not shown). In some embodiments, the pack of batteries comprises lithium-based batteries.

In some embodiments, a supercapacitor or ultracapacitor can be used to reduce lengthy boot-up when changing the battery packs. Instead of powering down head-mounted display unit 200, processor 233 may be configured to control the components of head-mounted display unit 200 to enter a low current standby mode. By powering off all components and peripherals, current may be reduced to the minimum, so as to enable the supercapacitor or ultracapacitor to retain the state of head-mounted display unit 200 for a sufficiently long time interval of switching the battery packs without the need for an additional battery for the standby mode.

In some embodiments, head-mounted display unit 200 may comprise various types of image sources, such as but not limited to OLED and liquid-crystal on silicon. These image sources may be combined with various sorts of optics (or optical setups), such as but not limited to visor-based, waveguide-based, and birdbath-based optical engines. For example, head-mounted display unit 200 may comprise a combination of OLED and visor, or a combination of liquid-crystal on silicon and waveguide sources. However, it is noted that all possible permutations of the above image source and optics may be applicable for head-mounted display unit 200.

FIG. 2B is a schematic pictorial illustration of an exemplary head-up display (HUD) 270, in accordance with one embodiment that can be used as the head-mounted display unit 28 in FIG. 1. HUD 270 also serves and is referred to herein as an HMD, and may replace, for example, head-mounted display unit 200 of FIG. 2A above.

In some embodiments, HUD 270 comprises an optics housing 274, which incorporates a camera 278. More specifically, camera 278 may comprise an RGB camera configured as an IR camera using a suitable filter and software or the camera 278 may comprise an infrared camera or an RGB-IR camera. In some embodiments, optics housing 274 comprises an infrared transparent window 282, and within the housing, e.g., behind the window, are mounted one or more infrared projectors 276.

In some embodiments, HUD 270 comprises a pair of AR displays 280 that are mounted on optics housing 274. In some embodiments, displays 280 may comprise, for example, an optical combiner, a waveguide, or a visor, as described in connection with FIG. 2A above.

In some embodiments, AR displays 280 allow surgeon 26 to view entities, such as part or all of a selected field-of-view (not shown) through AR displays 280, and which are also configured to present to the surgeon images that may be received from processing system 50 or any other information.

In some embodiments, HUD 270 comprises a processor 284, which operates elements of HUD 270 and is mounted in a processor housing 286. Processor 284 may communicate with processing system 50 via an antenna 288. In some embodiments, processor 284 may perform some of the functions performed by processing system 50. In some embodiments, processor 284 may completely replace processing system 50.

In some embodiments, HUD 270 comprises a flashlight 292, which is mounted on the front of HUD 270. Flashlight 292 is configured to direct a beam of visible spectrum light (e.g., wavelengths between about 350 nm and 800 nm or between about 300 nm and 900 nm) to selected objects, so that surgeon 26 or other wearer is able to clearly see the objects through displays 280.

In some embodiments, HUD 270 comprises a power source (e.g., a battery (not shown)), which is configured to supply power to several elements of HUD 270 via a battery cable input 296. The power source may additionally or alternatively include one or more capacitors, supercapacitors, or ultracapacitors.

In some embodiments, HUD 270 is held and gripped in place on the head of surgeon 26 using a head strap 290, and comprises a knob 294 that the surgeon 26 may use to adjust the head strap of HUD 270. The head strap 290 and knob 294 may together be referred to as an adjustable strap assembly.

In some embodiments, HUD 270 may comprise additional components, such as but not limited to components described in FIGS. 1 and 2A above. For example, HUD 270 may comprise an inertial measurement unit (IMU), which is configured to produce position signals indicative of the position and orientation of a tracking system reference point/origin of HUD 270. Moreover, HUD 270 may comprise IR LED projectors.

Additionally, or alternatively, flashlight 292 of HUD 270 may be coupled to housing 274 using a suitable detachable lighting fixture assembly (DLFA), which is configured to be attached to and detached from housing 274 and/or the upper bridge (not indicated by a numeral) of HUD 270, or any other suitable location of HUD 270. The ability to detach flashlight 292 reduces weight from HUD 270.

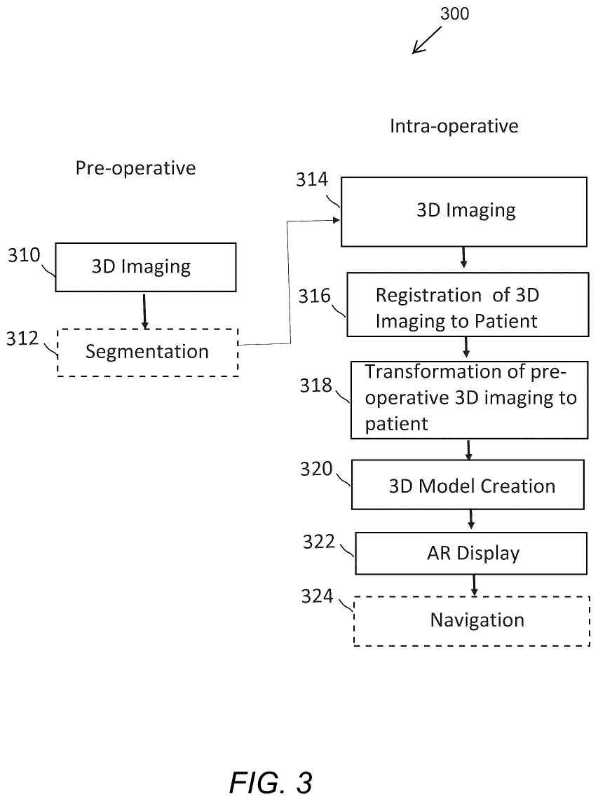

FIG. 3 is a flowchart of a registration and augmented reality visualization workflow 300 to facilitate augmented reality image-guided navigation using high-quality images. In this workflow, a patient (e.g., patient 20) undergoes a pre-operative 3D imaging scan (e.g., CT scan or MRI scan that can be converted to a CT-like scan) at step 310. At step 312, the 3D imaging scan (e.g., CT scan or MRI scan that can be converted to a CT-like scan) is stored in memory (e.g., imported into memory of a workstation) and optionally undergoes segmentation. The segmentation can be automatically performed by software or artificial intelligence (AI) techniques, such as by trained neural networks, and a user can make manual adjustments as needed during segmentation (e.g., using manual visualization and adjustment techniques). The segmentation may segment the 3D imaging into rigid bodies or anatomical units (e.g., individual vertebrae, such as L1, L2, L3, L4, L5, S1, S2, S3, C1, C2, C3, C4, T1, T2, T3, T4, etc., depending on the portion of the spine of interest or portion of the spine included in the imaging).

The workflow then enters the intra-operative phase. At step 314, one or more intra-operative 3D images are acquired using an intra-operative 3D imaging device (e.g., an intra-operative CT imaging device, such as a portable O-arm imaging device). The intra-operative 3D images are acquired in a way that allows registering the intra-operative 3D images to the patient. For example, the intra-operative 3D images may include images that include images of a fiducial reference marker (e.g., reference marker 38) attached to or placed on the patient 20 near a region of interest (e.g., a region where a medical procedure is being performed or will be performed).

At step 316, registration of the intra-operative 3D images to the patient based on the location and/or orientation of the fiducial reference marker (e.g., reference marker 38) in the intra-operative 3D images and the location and/or orientation of the fiducial reference marker (e.g., reference marker 38) based on images captured by an optical tracker or sensor (e.g., infrared camera or tracking sensor 34) of the head-mounted unit 28 (e.g., head-mounted display unit 200, HUD 270) is performed. Registration may also include use of a patient marker (e.g., patient marker 60) such as described in connection with FIG. 1 and in US Publication No. 2021/0161614), for example.

At step 318, a 3D-3D matching process is performed between the pre-operative 3D imaging and the intra-operative 3D images to determine a transformation of the pre-operative imaging to the patient such that the pre-operative imaging is now registered to the patient based on the registration of the intra-operative imaging to the patient and such that the intra-operative imaging (or 3D models generated therefrom) may be displayed in alignment with the patient anatomy based on the registration. This matching process may be performed globally or may be performed locally based on matching a rigid body (e.g. vertebra or other anatomical unit) identified during segmentation (at step 312) with a corresponding rigid body (e.g., vertebra or other anatomical unit) in the intra-operative 3D images. For example, a user (e.g., the surgeon or other clinical professional 26) generates an initial guess marking on the intra-operative 3D images by selecting one of the segmented vertebra or other bony portion, from the pre-operative 3D imaging and marking its position on the intra-operative 3D images. The initial guess marking could also be performed automatically by the processor(s). The matching may involve comparison of image similarity values (e.g., pixel value comparison), for example.

At step 320, a processor, upon execution of stored program instructions, creates a new virtual 3D model based on the pre-operative 3D imaging and the transformation determined at step 318. At step 322, an AR display of the virtual 3D model (and optionally additional 2D images based on the pre-operative and/or intra-operative 3D images (e.g., 2D slices of anterior-posterior views, lateral views, sagittal views)) is generated for display on the head-mounted unit 28.

At optional step 324, the user (e.g., the surgeon or other clinical professional 26) performs a medical procedure by navigation using the AR display generated at step 322. The navigation may include registration of one or more tools for use in the medical procedure by using the optical camera or sensor 34 of the head-mounted unit 28 to scan a tool marker 40 coupled to a respective one of the one or more tools 22 (e.g., via a tool adapter) as well as a patient marker 60 coupled to the patient at the region of interest (e.g., indirectly coupled to a clamp or percutaneous pin that is coupled to patient anatomy, such as bone of the patient, or directly coupled to the patient at the region of interest), such as described in connection with FIG. 1.

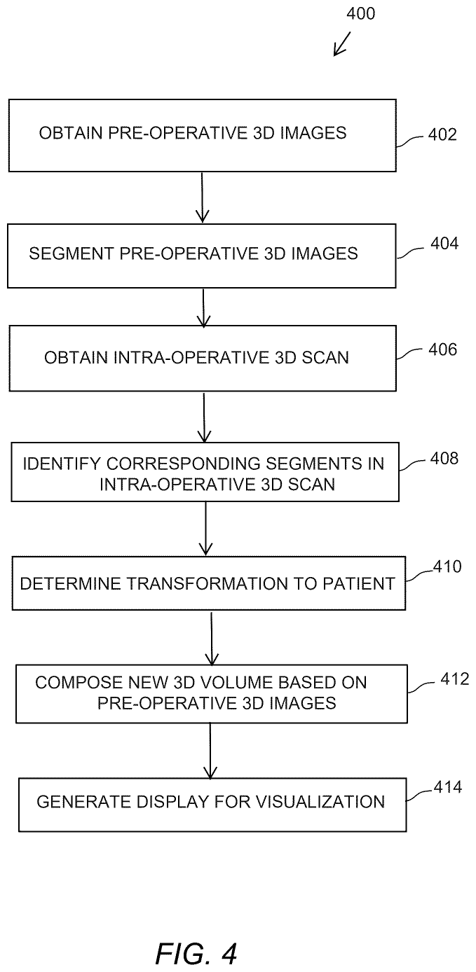

FIG. 4 is a flow-chart of a process 400 that can be performed by execution of stored program instructions using one or more processors (e.g., any of the processors described herein) to generate high-quality augmented reality images that can be used to facilitate navigation during a medical procedure by a surgeon or other clinical professional wearing a head-mounted display unit (e.g., head-mounted display unit 28) such that the surgeon or other clinical professional does not have to look away from the site of the medical procedure during the medical procedure.

At step 402, the one or more processors obtain pre-operative 3D images. The 3D images may be obtained from local memory or remotely over a communications network. The 3D images may be a 3D scan volume acquired by a CT scanner or imaging device, an MRI scanner, an ultrasound imaging device, a PET scanner or imaging device, or other 3D imaging device.

At step 404, the one or more processors segment the one or more pre-operative 3D images into segmented units (e.g., individual rigid bodies or anatomical units). The segmentation can be automatically performed by software or AI techniques, such as by trained neural networks, and a user can make manual adjustments as needed during segmentation (e.g., using manual visualization and adjustment techniques). For spinal applications, the segmentation may segment the 3D imaging into individual vertebrae, sacrum and ileum (such as individual vertebrae of the lumbar, thoracic, sacral or cervical sections of the spine, depending on the portion of the spine of interest or the portion of the spine included in the imaging). The segmented individual vertebrae may be labeled with an indicator of the level and number of the vertebra (e.g., L1, L2, L3, . . . , S1, S2, . . . , C1, C2, C3, . . . , T1, T2, . . . , etc.). The segmented individual vertebrae may be color-coded.

At step 406, the one or more processors obtain an intra-operative 3D scan. The intra-operative 3D scan may be a set of one or more 3D images acquired by an intra-operative CT imaging device, such as a portable cone-beam CT imaging device or an O-arm imaging device. The intra-operative 3D scan may be used to register the intra-operative images to the region of interest of the patient using the registration techniques described above and in US Publication No. 2021/0161614, previously incorporated by reference in its entirety.

At step 408, corresponding segments of the segmented units of the pre-operative 3D images are identified by the one or more processors in the intra-operative 3D scan. The identification may be performed using 3D-3D matching algorithms, for example. The matching may involve feature matching algorithms and/or comparison of image similarity values (e.g., pixel value comparison), for example.

At step 410, the one or more processors determine a transformation from the pre-operative images to the patient based on the matching and the previous registration performed at steps 406 and 408. The result is that the pre-operative images are now registered to the patient using the registration of the intra-operative images and pre-operative imaging (e.g., a 3D volume or 3D model generated from the pre-operative images) having a higher image quality can now be displayed aligned with the patient anatomy instead of images based on the lower image quality intra-operative images.

At step 412, the one or more processors compose a new 3D volume (e.g., virtual 3D model) based on the pre-operative 3D images and the determined transformation at step 410.

At step 414, the one or more processors generate an AR display for display on the head-mounted display unit 28 that includes the 3D volume. Based on the determined transformation, the 3D volume is displayed in alignment with the corresponding patient anatomy. The AR display may also include one or more 2D images that can be used to facilitate navigation during the medical procedure. The 2D images may be generated based on the intra-operative 3D scan or the pre-operative 3D scan. The AR display may also include virtual images of one or more tools or implements used in the medical procedure (e.g., screwdrivers, Jamshidi needles, screws, rods, plates, cages). The virtual images of the one or more tools or implements may be positioned based on registration performed using markers (e.g., markers 40, 60) coupled to the one or more tools and to the patient that are imaged using an optical camera or sensor (e.g., infrared sensor or visible wavelength optical camera), such as described in connection with FIG. 1.

### Example Clauses

Clause 1. Clause 1. A method for facilitating high image quality augmented-reality assisted navigation based using pre-operative three-dimensional (3D) imaging of at least a portion of an anatomy of a patient registered to intra-operative 3D imaging of at least the portion of the anatomy of the patient, the method comprising: obtaining one or more pre-operative 3D images of at least the portion of the anatomy of the patient; obtaining one or more intra-operative 3D images of at least the portion of the anatomy of the patient, the one or more intra-operative 3D images including a registration marker coupled to the patient adjacent the portion of the anatomy of the patient; registering the one or more intra-operative 3D images to the portion of the anatomy of the patient based on optical imaging of the registration marker and a patient marker coupled to the portion of the anatomy using an optical imaging device of a head-mounted display unit; matching corresponding features of the one or more pre-operative 3D images and the one or more intra-operative 3D images to determine a transformation between the one or more pre-operative 3D images and the portion of the anatomy of the patient; and generating an output for display on an augmented-reality display of the head-mounted display unit that is based on the one or more pre-operative 3D images.

Clause 2. The method of Clause 1, wherein the portion of the anatomy comprises a portion of a spine of the patient.

Clause 3. The method of any one of Clauses 1-2, wherein the one or more pre-operative 3D images comprise computed tomography images generated from one or more magnetic resonance images.

Clause 4. The method of any one of Clauses 1-3, wherein the display includes a virtual 3D model or mesh generated from the one or more pre-operative 3D images.

Clause 5. The method of any one of Clauses 1-4, wherein the augmented-reality display is a see-through stereoscopic display of the head-mounted display unit.

Clause 6. The method of Clause 5, wherein the head-mounted display unit comprises glasses.

Clause 7. The method of any one of Clauses 5-6, wherein the head-mounted display unit is an over-the-head mounted device.

Clause 8. The method of any one of Clauses 1-7, further comprising segmenting the one or more pre-operative 3D images into separate individual anatomical segments or rigid bodies.

Clause 9. The method of any one of Clauses 1-8, wherein the one or more pre-operative 3D images are obtained from local memory or over a communications network.

Clause 10. The method of any one of Clauses 1-9, wherein the portion of the anatomy of the patient is at least a portion of a spine.

Clause 11. A system for facilitating augmented-reality assisted navigation using pre-operative three-dimensional (3D) imaging of at least a portion of an anatomy of a patient registered to intra-operative 3D imaging of at least the portion of the anatomy of the patient, the system comprising: a wearable augmented reality display unit configured to provide an augmented reality display to facilitate navigation of tracked instruments by a wearer of the wearable augmented reality display unit; one or more processors communicatively coupled to non-transitory computer-readable storage media storing instructions that, when executed by the one or more processors, cause the one or more processors to: obtain one or more pre-operative 3D images of at least the portion of the anatomy of the patient; obtain one or more intra-operative 3D images of at least the portion of the anatomy of the patient, the one or more intra-operative 3D images including a registration marker coupled to the patient adjacent the portion of the anatomy of the patient; register the one or more intra-operative 3D images to the portion of the anatomy of the patient based on optical imaging of the registration marker and a patient marker coupled to the portion of the anatomy using an optical imaging device of a head-mounted display unit; match corresponding features of the one or more pre-operative 3D images and the one or more intra-operative 3D images to determine a transformation between the one or more pre-operative 3D images and the portion of the anatomy of the patient; and generate an output for display on an augmented-reality display of the head-mounted display unit that is based on the one or more pre-operative 3D images.

Clause 12. The system of Clause 11, wherein the portion of the anatomy comprises a portion of a spine of the patient.

Clause 13. The system of any one of Clauses 11-12, wherein the one or more pre-operative 3D images comprise computed tomography images or computed tomography-like images generated from one or more magnetic resonance images.

Clause 14. The system of any one of Clauses 11-13, wherein the display includes a virtual 3D model or mesh generated from the one or more pre-operative 3D images.

Clause 15. The system of any one of Clauses 11-14, wherein the augmented-reality display is a see-through stereoscopic display of the head-mounted display unit.

Clause 16. The system of Clause 15, wherein the head-mounted display unit comprises glasses.

Clause 17. The system of any one of Clauses 15-16, wherein the head-mounted display unit is an over-the-head mounted device.

Clause 18. The system of any one of Clauses 11-17, wherein the instructions are further configured to cause the one or more processors to segment the one or more pre-operative 3D images into separate individual anatomical segments or rigid bodies.

Clause 19. The system of any one of Clauses 11-18, wherein the one or more pre-operative 3D images are obtained from local memory or over a communications network.

Clause 20. The method of any one of Clauses 1-10, wherein the portion of the anatomy of the patient is at least a portion of a spine.

Clause 21. A method of facilitating augmented-reality assisted navigation using high quality images generated from pre-operative 3D imaging of at least a portion of a spine of a patient based on registration with lower quality intra-operative 3D imaging as described herein.

Clause 22. A system for facilitating augmented-reality assisted navigation using high quality images generated from pre-operative 3D imaging of at least a portion of a spine of a patient based on registration with lower quality intra-operative 3D imaging as described herein.

Clause 26. The use of any one of Clauses 1-20 for the treatment of a spine through a surgical intervention.

Clause 27. The use of any one of Clauses 1-20 for the treatment of an orthopedic joint through a surgical intervention, including, optionally, a shoulder, a knee, an ankle, a hip, or other joint.

Clause 28. The use of any one of Clauses 1-20 for the treatment of a cranium through a surgical intervention.

Clause 29. The use of any one of Clauses 1-20 for the treatment of a jaw through a surgical intervention.

Clause 30. The use of any one of Clauses 1-20 for the diagnosis of a spinal abnormality.

Clause 31. The use of any one of Clauses 1-20 for the diagnosis of a spinal injury.

Clause 32. The use of any one of Clauses 1-20 for the diagnosis of joint damage.

Clause 33. The use of any one of Clauses 1-20 for the diagnosis of an orthopedic injury.

Clause 34: A processing system, comprising: a memory comprising computer-executable instructions; and a processor configured to execute the computer-executable instructions and cause the processing system to perform a method in accordance with any one of Clauses 1-10.

Clause 35: A processing system, comprising means for performing a method in accordance with any one of Clauses 1-10.

Clause 36: A non-transitory computer-readable medium storing program code for causing a processing system to perform the steps of any one of Clauses 1-b 10.

Clause 37: A computer program product embodied on a computer-readable storage medium comprising code for performing a method in accordance with any one of Clauses 1-b 10.

### Conclusion and Terminology

While examples of the disclosed technique are given for a body portion containing spine vertebrae, the principles of the system, method, and/or disclosure may also be applied to other bones and/or body portions than the spine, including hip bones, pelvic bones, leg bones, arm bones, ankle bones, foot bones, shoulder bones, cranial bones, oral and maxillofacial bones, sacroiliac joints, etc.

The disclosed techniques are presented with relation to image-guided surgery systems or methods, in general, and accordingly, the disclosed technique of visualization of medical images should not be considered limited only to augmented reality systems and/or head-mounted systems. For example, the technique is applicable to the processing of images from different imaging modalities, as described above, for use in diagnostics.

The terms “top,” “bottom,” “first,” “second,” “upper,” “lower,” “height,” “width,” “length,” “end,” “side,” “horizontal,” “vertical,” and similar terms may be used herein; it should be understood that these terms have reference only to the structures shown in the figures and are utilized only to facilitate describing embodiments of the disclosure. Various embodiments of the disclosure have been presented in a range format. It should be understood that the description in range format is merely for convenience and brevity and should not be construed as an inflexible limitation on the scope of the disclosure. The ranges disclosed herein encompass any and all overlap, sub-ranges, and combinations thereof, as well as individual numerical values within that range. For example, description of a range such as from about 5 to about 30 degrees should be considered to have specifically disclosed subranges such as from 5 to 10 degrees, from 10 to 20 degrees, from 5 to 25 degrees, from 15 to 30 degrees etc., as well as individual numbers within that range (for example, 5, 10, 15, 20, 25, 12, 15.5 and any whole and partial increments therebetween). Language such as “up to,” “at least,” “greater than,” “less than,” “between,” and the like includes the number recited. Numbers preceded by a term such as “about” or “approximately” include the recited numbers. For example, “approximately 2 mm” includes “2 mm.” The terms “approximately”, “about”, and “substantially” as used herein represent an amount close to the stated amount that still performs a desired function or achieves a desired result.

In some embodiments, the system comprises various features that are presented as single features (as opposed to multiple features). For example, in one embodiment, the system includes a single HMD, a single camera, a single processor, a single display, a single marker, a single calibration jig, a single image, a single bead plate, a single imaging device, a single fluoroscope, etc. Multiple features or components are provided in alternate embodiments.

In some embodiments, the system comprises one or more of the following: means for imaging (e.g., a camera or MRI machine or CT machine), means for registration (e.g., adapters, markers, objects, cameras), means for fastening (e.g., anchors, adhesives, clamps, pins), means for segmentation (e.g., one or more neural networks), etc.

The processors described herein may include one or more central processing units (CPUs) or processors or microprocessors. The processors may be communicatively coupled to one or more memory units, such as random-access memory (RAM) for temporary storage of information, one or more read only memory (ROM) for permanent storage of information, and one or more mass storage devices, such as a hard drive, diskette, solid state drive, or optical media storage device. The processors (or memory units communicatively coupled thereto) may include modules comprising program instructions or algorithm steps configured for execution by the processors to perform any or all of the processes or algorithms discussed herein. The processors may be communicatively coupled to external devices (e.g., display devices, data storage devices, databases, servers, etc. over a network via a network communications interface.

In general, the algorithms or processes described herein can be implemented by logic embodied in hardware or firmware, or by a collection of software instructions, possibly having entry and exit points, written in a programming language, such as, for example, Python, Java, Lua, C, C #, or C++. A software module or product may be compiled and linked into an executable program, installed in a dynamic link library, or may be written in an interpreted programming language such as, for example, BASIC, Perl, or Python. It will be appreciated that software modules may be callable from other modules or from themselves, and/or may be invoked in response to detected events or interrupts. Software modules configured for execution on computing devices may be provided on a computer-readable medium, such as a compact disc, digital video disc, flash drive, or any other tangible medium. Such software code may be stored, partially or fully, on a memory device of the executing computing device, such as the computing system 50, for execution by the computing device. Software instructions may be embedded in firmware, such as an EPROM. It will be further appreciated that hardware modules may be comprised of connected logic units, such as gates and flip-flops, and/or may be comprised of programmable units, such as programmable gate arrays or processors. The modules described herein are preferably implemented as software modules, but may be represented in hardware or firmware. Generally, any modules or programs or flowcharts described herein may refer to logical modules that may be combined with other modules or divided into sub-modules despite their physical organization or storage.

The various features and processes described above may be used independently of one another or may be combined in various ways. All possible combinations and sub-combinations are intended to fall within the scope of this disclosure. In addition, certain method or process blocks or steps may be omitted in some implementations. The methods and processes described herein are also not limited to any particular sequence, and the blocks, steps, or states relating thereto can be performed in other sequences that are appropriate. For example, described blocks, steps, or states may be performed in an order other than that specifically disclosed, or multiple blocks or states may be combined in a single block or state. The example blocks, steps, or states may be performed in serial, in parallel, or in some other manner. Blocks, steps, or states may be added to or removed from the disclosed example embodiments. The example systems and components described herein may be configured differently than described. For example, elements may be added to, removed from, or rearranged compared to the disclosed example embodiments.

Any process descriptions, elements, or blocks in the flow diagrams described herein and/or depicted in the attached figures should be understood as potentially representing modules, segments, or portions of code which include one or more executable instructions for implementing specific logical functions or steps in the process.

It will be appreciated that the systems and methods of the disclosure each have several innovative aspects, no single one of which is solely responsible or required for the desirable attributes disclosed herein. The various features and processes described above may be used independently of one another or may be combined in various ways. The section headings used herein are merely provided to enhance readability and are not intended to limit the scope of the embodiments disclosed in a particular section to the features or elements disclosed in that section.

Certain features that are described in this specification in the context of separate embodiments also may be implemented in combination in a single embodiment. Conversely, various features that are described in the context of a single embodiment also may be implemented in multiple embodiments separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination. No single feature or group of features is necessary or indispensable to each and every embodiment.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

The terms “comprising,” “including,” “having,” and the like are synonymous and are used inclusively, in an open-ended fashion, and do not exclude additional elements, features, acts, operations, and so forth. In addition, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise.

What is claimed is:

1. A method for facilitating augmented-reality assisted navigation using pre-operative three-dimensional (3D) imaging of at least a portion of an anatomy of a patient registered to intra-operative 3D imaging of at least the portion of the anatomy of the patient, the method comprising:
obtaining one or more pre-operative 3D images of at least the portion of the anatomy of the patient;
obtaining one or more intra-operative 3D images of at least the portion of the anatomy of the patient, the one or more intra-operative 3D images including a registration marker coupled to the patient adjacent the portion of the anatomy of the patient;
registering the one or more intra-operative 3D images to the portion of the anatomy of the patient based on optical imaging of the registration marker and a patient marker coupled to the portion of the anatomy using an optical imaging device of a head-mounted display unit;
matching corresponding features of the one or more pre-operative 3D images and the one or more intra-operative 3D images to determine a transformation between the one or more pre-operative 3D images and the portion of the anatomy of the patient; and
generating an output for display on an augmented-reality display of the head-mounted display unit that is based on the one or more pre-operative 3D images.

2. The method of claim 1, wherein the portion of the anatomy comprises a portion of a spine of the patient.

3. The method of claim 1, wherein the one or more pre-operative 3D images comprise computed tomography images generated from one or more magnetic resonance images.

4. The method of claim 1, wherein the display includes a virtual 3D model or mesh generated from the one or more pre-operative 3D images.

5. The method of claim 1, wherein the augmented-reality display is a see-through stereoscopic display of the head-mounted display unit.

6. The method of claim 5, wherein the head-mounted display unit comprises glasses.

7. The method of claim 5, wherein the head-mounted display unit is an over-the-head mounted device.

8. The method of claim 1, further comprising segmenting the one or more pre-operative 3D images into separate individual anatomical segments or rigid bodies.

9. The method of claim 1, wherein the one or more pre-operative 3D images are obtained from local memory or over a communications network.

10. The method of claim 1, wherein the portion of the anatomy of the patient is at least a portion of a spine.

11. A system for facilitating augmented-reality assisted navigation using pre-operative three-dimensional (3D) imaging of at least a portion of an anatomy of a patient registered to intra-operative 3D imaging of at least the portion of the anatomy of the patient, the system comprising:
a wearable augmented reality display unit configured to provide an augmented reality display to facilitate navigation of tracked instruments by a wearer of the wearable augmented reality display unit;
one or more processors communicatively coupled to non-transitory computer-readable storage media storing instructions that, when executed by the one or more processors, cause the one or more processors to:
obtain one or more pre-operative 3D images of at least the portion of the anatomy of the patient;
obtain one or more intra-operative 3D images of at least the portion of the anatomy of the patient, the one or more intra-operative 3D images including a registration marker coupled to the patient adjacent the portion of the anatomy of the patient;
register the one or more intra-operative 3D images to the portion of the anatomy of the patient based on optical imaging of the registration marker and a patient marker coupled to the portion of the anatomy using an optical imaging device of a head-mounted display unit;
match corresponding features of the one or more pre-operative 3D images and the one or more intra-operative 3D images to determine a transformation between the one or more pre-operative 3D images and the portion of the anatomy of the patient; and
generate an output for display on an augmented-reality display of the head-mounted display unit that is based on the one or more pre-operative 3D images.

12. The system of claim 11, wherein the portion of the anatomy comprises a portion of a spine of the patient.

13. The system of claim 11, wherein the one or more pre-operative 3D images comprise computed tomography images or computed tomography-like images generated from one or more magnetic resonance images.

14. The system of claim 11, wherein the display includes a virtual 3D model or mesh generated from the one or more pre-operative 3D images.

15. The system of claim 11, wherein the augmented-reality display is a see-through stereoscopic display of the head-mounted display unit.

16. The system of claim 15, wherein the head-mounted display unit comprises glasses.

17. The system of claim 15, wherein the head-mounted display unit is an over-the-head mounted device.

18. The system of claims 11, wherein the instructions, when executed by the one or more processors, further cause the one or more processors to segment the one or more pre-operative 3D images into separate individual anatomical segments or rigid bodies.

19. The system of claim 11, wherein the instructions, when executed by the one or more processors, further cause the one or more processors to obtain the one or more pre-operative 3D images from a local memory or over a communications network.

20. The system of claim 11, wherein the portion of the anatomy of the patient is at least a portion of a spine.
